(19) United States
(12) Patent Application Publication
(10) Pub. No.: US 2023/0383350 A1
(43) Pub. Date: Nov. 30, 2023

(54) METHOD FOR PREDICTING THE RESPONSE TO TNF INHIBITORS

(71) Applicants: INSTITUT NATIONAL DE LA SANTE ET DE LA RECHERCHE MEDICALE, Paris (FR); CENTRE HOSPITALIER UNIVERSITAIRE DE TOULOUSE, Toulouse (FR); CENTRE NATIONAL DE LA RECHERCHE SCIENTIFIQUE (CNRS), Paris (FR); UNIVERSITE PAUL SABATGIER TOULOUSE III, Toulouse (FR)

(72) Inventors: Jean-luc DAVIGNON, Cedex 09 Marseille (FR); Yannick DEGBOE, Cedex 3 TOULOUSE (FR); Katy DIALLO, Versailles (FR); Arnaud CONSTANTIN, Cedex 3 TOULOUSE (FR); Benjamin RAUWEL, Buzet sur Tarn (FR); Jean-Frederic BOYER, Cedex 3 TOULOUSE (FR); Michel BARON, Cedex 3 TOULOUSE (FR)

(21) Appl. No.: 18/249,756

(22) PCT Filed: Oct. 19, 2021
(86) PCT No.: PCT/EP2021/078973

§ 371 Date: Apr. 20, 2023

(30) Foreign Application Priority Data
Oct. 20, 2020 (EP) 20306247.6

(51) Int. Cl. C12Q 1/6883; C07K 16/24; A61K 38/17; A61P 19/02

(52) U.S. Cl. CPC C12Q 1/6883; C07K 16/241; A61K 38/1793; A61P 19/02; C12Q 2600/158; C12Q 2600/106

(57) ABSTRACT

Rheumatoid arthritis (RA) is the most prevalent chronic autoimmune inflammatory rheumatism. Its pathophysiology is largely dependent on TNF. Severe RA as well as several other inflammatory and autoimmune diseases are treated with TNF inhibitors (TNFi). However, to date only 30-50% achieve low disease activity or remission with this treatment regimen and some patients experience secondary non-response or relapse. Herein, the inventors evaluated by RT-qPCR the mRNA expression of CD36, which was already described to be regulated by TNFi⁵, some specific NRF2 target genes (FBX030, GABARA, LBR, MAFG, OSGIN1, HMOX1), which play a role in the anti-oxidative stress response or anti-inflammatory pathway, and the expression of CSMD1, an anti-inflammatory gene that we observed as up-regulated by all TNFi. Interestingly, they observed 2 different subsets of healthy donors: (i) donors in which TNFi stimulation increased mRNA of target genes in macrophages and (ii) conversely donors with no significant upregulation in transcription of these target genes. Then they classified donors two different status, “activators” or “non-activators” of tmTNF reverse signaling after TNFi stimulation, which correlates to clinically responder and non-responders to TNFi. Thus, based on all these observations, they developed an in vitro method for predicting the response to TNF inhibitors in patient in need thereof.

### FIELD OF THE INVENTION

The invention relates to the field of medicine, and more particularly to a method for predicting the clinical response to anti-TNF antibody in patient in need thereof.

### BACKGROUND OF THE INVENTION

Rheumatoid arthritis (RA) is the most prevalent chronic autoimmune inflammatory rheumatism. Its pathophysiology is largely dependent on TNF. Severe RA as well as several other inflammatory and autoimmune diseases are treated with TNF inhibitors (TNFi). Among TNFi, three different types of molecules with different clinical and biological properties are currently available: 3 monoclonal antibodies (adalimumab, infliximab, golimumab), 1 Fab coupled to pegol (certolizumab) and 1 soluble receptor fusion protein (etanercept).

TNFi have been a “game-changer”, especially in the prognosis of severe RA. Consequently, they are widely used in this indication. However, to date only 30-50% achieve low disease activity or remission with this treatment regimen and some patients experience secondary non-response or relapse. Improving the treatment of RA and other inflammatory diseases is a major objective of modern therapy. In a personalized medicine perspective, identifying patients with potential high therapeutical success rate is critical. TNFis appear to have different mechanisms of action which may explain their disparate responses in RA and other diseases^{1,2}.

TNF is generated as a transmembrane precursor (tmTNF). Its cleavage by the TACE protease (TNF-Alpha Converting Enzyme) leads to release of a soluble form (sTNF) which can interact with its receptor during inflammation. In addition to its classic interaction with its receptor, it has been shown that tmTNF can act itself as a receptor which transmits reverse signaling in cells^{3,4}.

Our laboratory has shown that the activation of this reverse signaling by a TNFi molecule involves in part the nuclear factor NRF2⁵. Given the oxidative burden associated with RA, and in general with inflammatory diseases, an anti-oxidant effect of reverse signaling could therefore play a crucial role in efficacy of TNFi treatment. Indeed, NRF2 is one of the critical regulators which promotes the transcription of a wide variety of antioxidant genes (such as HMOX1) and plays a key role in controlling inflammation, by antagonizing NF-kB for example⁶. The inventors were able to show in a transgenic mouse model, which do not express TNF receptors (TNFR1/R2) and express only the transmembrane form of TNF, that reverse signaling plays an important role in the therapeutic response to TNFi during arthritis⁷. In addition, it has also been shown that the clinical response to TNFi biotherapy in patients with RA does not depend on the serum concentration of soluble TNF⁸. TNFi molecules have similar affinity and ability to neutralize soluble TNF⁹, which suggests that the intra-individual variability of response to the different TNFi molecules observed depends on another mechanism. However, many other signaling pathways downstream from this tmTNF remain to be discovered. Although mentioned in numerous publications, the reverse signaling of tmTNF, its molecular mechanisms and its importance in pathophysiology are not fully deciphered.

### SUMMARY OF THE INVENTION

The present invention relates to a method for predicting the clinical response to anti-TNF antibody in patient in need thereof. In particular, the present invention is defined by the claims.

### DETAILED DESCRIPTION OF THE INVENTION

The inventors analyzed the transcriptional activity of NRF2 in macrophages of healthy donors after TNFi stimulation. To this end, they evaluated by RT-qPCR the mRNA expression of CD36, which was already described to be regulated by TNFi⁵, some specific NRF2 target genes (FBX030, GABARA, LBR, MAFG, OSGIN1, HMOX1), which play a role in the anti-oxidative stress response or anti-inflammatory pathway, and the expression of CSMD1, an anti-inflammatory gene that we observed as up-regulated by all TNFi. Interestingly, they observed 2 different subsets of healthy donors: (i) donors in which TNFi stimulation increased mRNA of target genes in macrophages and (ii) conversely donors with no significant upregulation in transcription of these target genes. Then they classified donors into two different statuses, “activators” or “non-activators” of tmTNF reverse signaling after TNFi stimulation, which correlate to clinically responders and non-responders to TNFi.

Based on all these observations, the inventors developed a protocol to classify donors or patients into activators or non-activators (i.e responder or non-responder) of tmTNF reverse signaling after TNFi stimulation. After 16 hours of TNFi treatment, we first analyzed the CD36 mRNA ratio (Treated vs NT). If this ratio is lower than a reference value (lower than 1 after application of our CV), then the donor/patient is classified as non-activator. If this ratio is upper than a reference value (upper than 1 after application of our CV), then they analyzed the modulation of 6 NRF2 target genes (GABARA, FBX030, HMOX1, MAFG, LBR, OSGIN1).

Accordingly, in a first aspect, the invention relates to an in vitro method for classifying anti-TNF antibody activator and non-activator patient in need thereof, comprising the steps of:
i) determining, in a sample obtained from the patient, the expressions levels of CD36 gene with and without stimulation with anti-TNF antibody,
ii) calculating the ratio between the expressions levels of CD36 with and without stimulation with anti-TNF antibody determined at step i),
iii) comparing the ratio determined at step ii) with a reference value,
iv) concluding that the patient is a non-activator when the ratio determined at step ii) is lower than the reference value.

In some embodiment, when the ratio determined at step i) is higher than the reference value, the following steps are added:
v) determining in the sample obtained from the patient, the expressions levels of at least two genes selected from the group consisting in: FBX030, GABARA, LBR, MAFG, OSGIN1, HMOX1 and CSMD1 with and without stimulation with anti-TNF antibody,
vi) calculating for each gene determined at step v) the ratio between the expression level of gene with and without stimulation with anti-TNF antibody, and
vii) concluding that the patient is a non-activator when at least two ratio determined at step vi) are lower than the reference value or concluding that the patient is an activator when at least one of ratio determined at step iv) are higher than the reference value.

Thus, the invention relates to an in vitro method for classifying anti-TNF antibody activator and non-activator patient in need thereof, comprising the steps of:
i) determining, in a sample obtained from the patient, the expressions levels of CD36 gene with and without stimulation with anti-TNF antibody,
ii) calculating the ratio between the expressions levels of CD36 with and without stimulation with anti-TNF antibody determined at step i),
iii) comparing the ratio determined at step ii) with a reference value,
iv) concluding that the patient is a non-activator when the ratio determined at step ii) is lower than the reference value, or
v) determining in the sample obtained from the patient, the expressions levels of at least two genes selected from the group consisting in: FBX030, GABARA, LBR, MAFG, OSGIN1, HMOX1 and CSMD1 with and without stimulation with anti-TNF antibody, when the expression level of CD36 determined at step i) is higher than the reference value; and
vi) calculating for each gene determined at step v), the ratio between the expression level of gene with and without stimulation with anti-TNF antibody, and
vii) concluding that the patient is a non-activator when at least two ratios determined at step vi) are lower than the reference value or concluding that the patient is an activator when at least one of ratio determined at step iv) are higher than the reference value.

In some embodiment, the expression level of 2, 3, 4, 5, 6 or 7 genes selected from the group consisting in: FBX030, GABARA, LBR, MAFG, OSGIN1, HMOX1 and CSMD1 are determined with and without stimulation with anti-TNF antibody in step v).

In some embodiment, it will be concluded at step vii) that patient is a non-activator when 2, 3, 4, 5, 6 or 7 ratio determined at step vi) is lower than the reference value or concluding that the patient is an activator when 2, 3, 4, 5, 6 or 7 ratio determined at step vi) are higher than the reference value.

In some embodiment, it will be concluded at step vii) that patient is a non-activator when more than 50% of the ratios determined at step vi) are lower than the reference value or concluding that the patient is an activator when at least 50% of the ratios determined at step vi) are higher than the reference value.

As used herein, the term “at least 50% of the ratios” refers to 1 ratio when there are 2 ratios determined at step vi), at least 2 ratios when there are 3 or 4 ratios determined at step vi) and refers to at least 3 ratios when there are 5 or 6, and refers to at least 4 ratios when there are 7 ratios determined at step vi).

As used herein, the term “more than 50% of the ratios” refers to at least 2 ratios when there are 2, 3 ratios determined at step vi), at least 3 ratios when there are 4 or 5 ratios determined at step vi) and refers to at least 4 ratios when there are 6 or 7 ratios determined at step vi).

In other word, it will be concluded at step vii) that patient is a non-activator when 50% of the ratios more one determined at step vi) are lower than the reference value or concluding that the patient is an activator when at least 50% of the ratios determined at step vi) are higher than the reference value.

According to the invention, the patient is considered as an activator when at least 50% of the ratios determined at step vi) are higher than the reference value and more than 50% of the ratios determined at step vi) are lower than the reference value.

Thus, the invention relates to an in vitro method for classifying anti-TNF antibody activator and non-activator patient in need thereof, comprising the steps of:
i) determining, in a sample obtained from the patient, the expressions levels of CD36 gene with and without stimulation with anti-TNF antibody,
ii) calculating the ratio between the expressions levels of CD36 with and without stimulation with anti-TNF antibody determined at step i),
iii) comparing the ratio determined at step ii) with a reference value,
iv) concluding that the patient is a non-activator when the ratio determined at step ii) is lower than the reference value, or
v) determining in the sample obtained from the patient, the expressions levels of 2, 3, 4, 5, 6 or 7 genes selected from the group consisting in: FBX030, GABARA, LBR, MAFG, OSGIN1, HMOX1 and CSMD1 with and without stimulation with anti-TNF antibody, when the expression level of CD36 determined at step i) is higher than the reference value; and vi) calculating for each gene determined at step v) the ratio between the expression level of gene after and without stimulation with anti-TNF antibody, and
vii) concluding that the patient is a non-activator when more than 50% of the ratios determined at step vi) is lower than the reference value or concluding that the patient is an activator when at least 50% of the ratios determined at step iv) is higher than the reference value.

In some embodiment, the expression level of FBX030, GABARA, LBR, MAFG, OSGIN1, and HMOX1 are determined with and without stimulation with anti-TNF antibody in step v).

Thus, the invention relates to an in vitro method for classifying anti-TNF antibody activator and non-activator patient in need thereof, comprising the steps of:
i) determining, in a sample obtained from the patient, the expressions levels of CD36 gene with and without stimulation with anti-TNF antibody,
ii) calculating the ratio between the expressions levels of CD36 with and without stimulation with anti-TNF antibody determined at step i),
iii) comparing the ratio determined at step ii) with a reference value,
iv) concluding that the patient is a non-activator when the ratio determined at step ii) is lower than the reference value, or
v) determining in the sample obtained from the patient, the expressions levels of FBX030, GABARA, LBR, MAFG, OSGIN1, and HMOX1, with and without stimulation with anti-TNF antibody, when the expression level of CD36 determined at step i) is higher than the reference value; and
vi) calculating for each gene determined at step v) the ratio between the expression level of gene after and without stimulation with anti-TNF antibody, and
vii) concluding that the patient is a non-activator when 4, 5 or 6 ratios determined at step vi) is lower than the reference value or concluding that the patient is an activator when 3, 4, 5 or 6 of ratios determined at step iv) is higher than the reference value.

In some embodiment, the expression level of FBX030, GABARA, LBR, MAFG, OSGIN1, HMOX1 and CSMD1 are determined with and without stimulation with anti-TNF antibody in step v).

Thus, the invention relates to an in vitro method for classifying anti-TNF antibody activator and non-activator patient in need thereof, comprising the steps of:
i) determining, in a sample obtained from the patient, the expressions levels of CD36 gene with and without stimulation with anti-TNF antibody,
ii) calculating the ratio between the expressions levels of CD36 with and without stimulation with anti-TNF antibody determined at step i),
iii) comparing the ratio determined at step ii) with a reference value,
iv) concluding that the patient is a non-activator when the ratio determined at step ii) is lower than the reference value, or
v) determining in the sample obtained from the patient, the expressions levels of FBX030, GABARA, LBR, MAFG, OSGIN1, HMOX1 and CSMD1, with and without stimulation with anti-TNF antibody, when the expression level of CD36 determined at step i) is higher than the reference value; and
vi) calculating for each gene determined at step v) the ratio between the expression level of gene after and without stimulation with anti-TNF antibody, and
vii) concluding that the patient is a non-activator when 4, 5, 6 or 7 ratios determined at step vi) is lower than the reference value or concluding that the patient is an activator when 4, 5, 6 or 7 ratios determined at step iv) is higher than the reference value.

According to the invention, the ratio for each gene is calculated according to the following formula:

$$Ratio R = \frac{\begin{matrix}Expression of the gene with stimulation with \\ anti - TNF antibody\end{matrix}}{\begin{matrix}Expression of the gene without stimulation with \\ anti - TNF antibody\end{matrix}}$$

As used herein, the term “anti-TNF antibody” for “anti-Tumor necrosis factor antibody” has its general meaning in the art and refers to an antibody that suppresses the physiologic response to tumor necrosis factor (TNF), which is part of the inflammatory response. Tumor necrosis factor (TNF) plays a central role in the pathogenesis of several inflammatory conditions, including rheumatoid arthritis (RA). TNF is made intracellularly, mainly by activated macrophages. The precursor TNF is converted to soluble TNF after proteolysis by the TNF-converting enzyme (TACE or ADAM-17). This soluble TNF then oligomerizes and forms the biologically active homotrimer TNF. There are two types of TNF, which are very closely related, TNF-alpha and TNF-beta (also known as lymphotoxin alpha). The activities of both TNFs are mediated through binding to the TNF receptors I and II (TNFRI and TNFRII).

According to the invention, anti-TNF antibody include anti-TNF monoclonal antibody such as adalimumab, golimumab, and infliximab.

As used herein the term “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). The terms “monoclonal antibody”, “monoclonal Ab”, “monoclonal antibody composition”, “mAb”, or the like, as used herein refer to a preparation of antibody molecules of single molecular composition. A monoclonal antibody composition displays a single binding specificity and affinity for a particular epitope. In some embodiment, the anti-TNF monoclonal antibody can be a chimeric antibody, particularly a chimeric mouse/human antibody, or a humanized antibody.

In some embodiment, the anti-TNF antibody is a monoclonal antibody.

In some embodiment, the anti-TNF monoclonal antibody is adalimumab, golimumab or infliximab.

Accordingly to the invention, the term “activator” refers to a patient whom the tmTNF reverse signaling have been activated after anti-TNF antibody stimulation.

Accordingly to the invention, the term “non-activator” refers to a patient whom the tmTNF reverse signaling have not been activated after anti-TNF antibody stimulation.

Accordingly to the invention, the term activator correspond to a patient who will respond to anti-TNF antibody and the term non-activator correspond to a patient who will not respond to anti-TNF antibody.

Thus, the invention relates to an in vitro method for predicting the clinical response to anti-TNF antibody wherein it is concluded that the patient identified as activators according to the invention will respond to anti-TNF antibody and the patient identified as non-activators according to the invention will not respond to anti-TNF antibody.

In other words the invention relates to an in vitro method for predicting the clinical response to anti-TNF antibody in patient in need thereof, comprising the steps of:
i) determining, in a sample obtained from the patient, the expressions levels of CD36 gene with and without stimulation with anti-TNF antibody,
ii) calculating the ratio between the expressions levels of CD36 with and without stimulation with anti-TNF antibody determined at step i),
iii) comparing the ratio determined at step ii) with a reference value,
iv) concluding that the patient will not respond to anti-TNF antibody when the ratio determined at step ii) is lower than the reference value, or
v) when the expression level of CD36 determined at step i) is higher than the reference value, determining in the sample obtained from the patient, the expressions levels of at least two genes selected from the group consisting in: FBX030, GABARA, LBR, MAFG, OSGIN1, HMOX1 and CSMD1 with and without stimulation with anti-TNF antibody, and
vi) calculating for each gene determined at step v), the ratio between the expression level of gene with and without stimulation with anti-TNF antibody, and
vii) concluding that the patient will not respond to anti-TNF antibody when more than 50% of the ratios determined at step vi) are lower than the reference value or concluding that the patient will respond when at least 50% of the ratios determined at step iv) are higher than the reference value.

As used herein, the term “patient” refers to any mammals, such as a rodent, a feline, a canine, and a primate. Particularly, in the present invention, the term “patient” refers to a human in need of TNF inhibitors therapy.

In some embodiment, the patient suffers from inflammatory disease.

As used herein, the expression “inflammatory disease” is used herein in the broadest sense and includes all diseases and pathological conditions having etiologies associated with a systemic or local abnormal and/or uncontrolled inflammatory response. For instance, over-expression of proinflammatory cytokines without proper controls leads to a variety of inflammatory diseases and disorders. This term includes autoimmune inflammatory disease, acute inflammatory diseases and chronic inflammatory diseases.

In particular, the above-mentioned inflammatory diseases may be one or more selected from the group consisting of asthma, preperfusion injury, transplant rejection, sepsis, septic shock, arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis, x-linked hyper IgM syndrome, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoriti s, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal gammopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis (e.g. chronic pancreatitis), polyradiculitis acuta, pyoderma gangrenosum, Quervain's thyreoiditis, acquired splenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, nonalcoholic fatty liver disease and endometriosis.

In some embodiment, the patient suffers from chronic autoimmune inflammatory rheumatism.

In some embodiment, the patient suffers from rheumatoid arthritis, Crohn's disease, ankylosing spondylitis or psoriatic arthritis.

In some embodiment, the patient have been previously treated with methotrexate.

In some embodiment, the patient have been previously treated with methotrexate and low dose of corticosteroid.

In some embodiment, the patient have been previously treated with methotrexate and less than 10 mg of corticosteroid.

As used herein, the term “methotrexate” has its general meaning in the art and refers to an antimetabolite decreasing the activity of the immune system and thus used to treat autoimmune inflammatory disease.

As used herein, the term “corticosteroid” has its general meaning in the art and refers to a class of steroid hormones that are produced in the adrenal cortex of vertebrates, as well as the synthetic analogues of these hormones.

As used herein, the term “sample” refers to any substance of biological origin. Examples of samples includes, but are not limited to blood, tumor, saliva, urine, cerebrospinal fluids, or any of other biological fluids or tissues. As used herein “blood” includes whole blood, plasma, peripheral-blood, peripheral blood mononuclear cell (PBMC), lymp sample, serum, circulating cells, constituents, or any derivative of blood. In particular embodiment, the sample is a blood sample, and more particularly peripheral blood mononuclear cell (PBMC).

In some embodiment, the sample is a CD14+ monocytes sample. As used herein, the term “CD14+ monocytes” refers to immature phagocytic cells expressing CD14 and circulating in the blood stream of the subject. CD14 belongs to the family of lipopolysaccharide (LPS) receptor antigens and is strongly expressed on the majority of Monocytes. Typically, the CD14+ monocytes are isolated from the blood sample using a filter and/or a marker based method.

In some embodiment, the sample is a macrophage sample. As used herein, the term “macrophage” has its general meaning in the art and refers to specialised cells involved in the detection, phagocytosis and destruction of bacteria and other harmful organisms. Macrophage can be differentiated from purified CD14+ monocytes from healthy donors in presence of GM-CSF prior stimulation by CZP in vitro.

In some embodiment, the sample has been stimulated with and without TFN inhibitors during 14, 15, 16, 17, 18, 19, 20, 21, 22, 23, 24, 25, 26, 27, 28, 29 or 30 hours.

In some embodiment, the sample has been stimulated with TFN inhibitors during 16 hours.

In some embodiment, the sample is CD14+ monocytes purified from blood sample and has been stimulated with TFN inhibitors.

In some embodiment, the expression level of gene are determined after 14, 15, 16, 17, 18, 19, 20, 21, 22, 23, 24, 25, 26, 27, 28, 29 or 30 hours of stimulation with and without TNF inhibitor in step i) and/or step v).

In some embodiment, the expression level of gene are determined after 16 hours of stimulation with and without TNF inhibitor in step i) and/or step v).

As used herein, the term “CD36” for “Cluster of differentiation 36”, also known as “platelet glycoprotein 4, fatty acid translocase” has its general meaning in the art and refers to the gene encoding for the fourth major glycoprotein of the platelet surface which serves as a receptor for thrombospondin in platelets and various cell lines. Its Entrez reference is 948.

As used herein, the term “FBX030” for “F-Box protein 30” has its general meaning in the art and refers to the gene encoding for a member of the F-box protein family which is characterized by an approximately 40 amino acid motif, the F-box. Its Entrez reference is 84085.

As used herein, the term “GABARA” for “Gamma-aminobutyric acid receptor-associated” has its general meaning in the art and refers to the gene encoding for a gamma-aminobutyric acid A receptors [GABA(A) receptors], a ligand-gated chloride channels that mediate inhibitory neurotransmission. Its Entrez reference is 11337.

As used herein, the term “LBR” for “Lamin-B receptor” has its general meaning in the art and refers to the gene encoding for a protein belonging to the ERG4/ERG24 family. This protein localizes to the inner membrane of the nuclear envelope and anchors the lamina and the heterochromatin to the membrane. Its Entrez reference is 3930.

As used herein, the term “MAFG” for “transcription factor MafG” has its general meaning in the art and refers to the gene encoding for a bZip Maf transcription factor protein. MafG is one of the small Maf proteins, which are basic region and leucine zipper (bZIP)-type transcription factors. Its Entrez reference is 4097.

As used herein, the term “OSGIN1” for “Oxidative Stress Induced Growth Inhibitor 1” has its general meaning in the art and refers to the gene encoding for an oxidative stress response protein that regulates cell death. Expression of the gene is regulated by p53 and is induced by DNA damage. The protein regulates apoptosis by inducing cytochrome c release from mitochondria. Its Entrez reference is 29948.

As used herein, the term “HMOX1” for “Heme Oxygenase 1” has its general meaning in the art and refers to the gene encoding for an essential enzyme in heme catabolism, which cleaves heme to form biliverdin, which is subsequently converted to bilirubin by biliverdin reductase, and carbon monoxide, a putative neurotransmitter. Its Entrez reference is 3162.

As used herein, the term “CSMD1” for “CUB And Sushi Multiple Domains 1” has its general meaning in the art and refers to the gene encoding for protein containing 14 N-terminal CUB domains that are separated from each other by a Sushi domains followed by an additional 15 tandem Sushi domain segment. Its Entrez reference is 64478.

As used herein, the term “expression level” refers, e.g., to a determined level of expression of gene of interest. The expression level of expression indicates the amount of expression product in a sample. The expression product of a gene of interest can be the ribonucleic acid of interest itself, a nucleic acid transcribed or derived therefrom, or the a polypeptide or protein derived therefrom.

Measuring the expression level of the genes listed above can be done by measuring the gene expression level of these genes and can be performed by a variety of techniques well known in the art.

Typically, the expression level of a gene may be determined by determining the quantity of mRNA. Methods for determining the quantity of mRNA are well known in the art. For example, the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence-based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labelled nucleic acid molecule provides an indicator of the presence or concentration of a target nucleic acid sequence (e.g., genomic target nucleic acid sequence) (to which the labelled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilb ene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912.

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649,138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can he produced that are identifiable based on their different spectral characteristics. For example, semiconductor nanocrystals can he produced that emit light of different colors based on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlsbad, Calif.).

Additional labels include, for example, radioisotopes (such as 3 H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labelled probe specifically hybridizable or specific for the target nucleic acid sequence (e.g., genomic target nucleic acid sequence). The slides are optionally pre-treated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 0.1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In another embodiment, the expression level is determined by metabolic imaging (see for example Yamashita T et al., Hepatology 2014, 60:1674-1685 or Ueno A et al., Journal of hepatology 2014, 61:1080-1087).

Expression level of a gene may be expressed as absolute expression level or normalized expression level. This normalization allows the comparison of the expression level in one sample, e.g., a patient sample, to another sample, or between samples from different sources. In a particular embodiment, expression levels of gene are normalized by comparing its expression after being stimulated with TNF inhibitor to the expression of said gene without stimulation with TNF inhibitor.

According to the invention, the expression level of the genes listed above may also be measured by measuring the protein expression level encoding by said genes and can be performed by a variety of techniques well known in the art.

Typically protein expression level may be measured for example by capillary electrophoresis-mass spectroscopy technique (CE-MS), flow cytometry, mass cytometry or ELISA performed on the sample.

In the present application, the “level of protein” or the “protein level expression” means the quantity or concentration of said protein. In particular embodiment, the protein is expressed at the cell surface for markers whose function is linked to their correct plasma membrane expression or total expression for markers whose function is not limited to membrane expression. In still another embodiment, the “level of protein” means the quantitative measurement of the proteins expression relative to a negative control.

Such methods comprise contacting a sample with a binding partner capable of selectively interacting with proteins present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, capillary electrophoresis-mass spectroscopy technique (CE-MS). etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against the proteins to be tested. A sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule is added. The secondary binding molecule is allowed to react with any captured sample marker protein, the plate is washed and the presence of the secondary binding molecule is detected using methods well known in the art.

Particularly, a mass spectrometry-based quantification methods may be used. Mass spectrometry-based quantification methods may be performed using either labelled or unlabelled approaches [DeSouza and Siu, 2012]. Mass spectrometry-based quantification methods may be performed using chemical labeling, metabolic labeling or proteolytic labeling. Mass spectrometry-based quantification methods may be performed using mass spectrometry label free quantification, a quantification based on extracted ion chromatogram (EIC) and then profile alignment to determine differential level of polypeptides.

Particularly, a mass spectrometry-based quantification method particularly useful can be the use of targeted mass spectrometry methods as selected reaction monitoring (SRM), multiple reaction monitoring (MRM), parallel reaction monitoring (PRM), data independent acquisition (DIA) and sequential window acquisition of all theoretical mass spectra (SWATH) [Moving target Zeliadt N 2014 The Scientist; Liebler Zimmerman Biochemistry 2013 targeted quantitation pf proteins by mass spectrometry; Gallien Domon 2015 Detection and quantification of proteins in clinical samples using high resolution mass spectrometry. Methods v81 p15-23; Sajic, Liu, Aebersold, 2015 Using data-independent, high-resolution mass spectrometry in protein biomarker research: perspectives and clinical applications. Proteomics Clin Appl v9 p 307-21].

Particularly, the mass spectrometry-based quantification method can be the mass cytometry also known as cytometry by time of flight (CYTOF) (Bandura D R, Analytical chemistry, 2009).

Particularly, the mass spectrometry-based quantification is used to do peptide and/or protein profiling can be use with matrix-assisted laser desorption/ionisation time of flight (MALDI-TOF), surface-enhanced laser desorption/ionization time of flight (SELDI-TOF; CLINPROT) and MALDI Biotyper apparatus [Solassol, Jacot, Lhermitte, Boulle, Maudelonde, Mangé 2006 Clinical proteomics and mass spectrometry profiling for cancer detection. Journal: Expert Review of Proteomics V3, 13, p 311-320; FDA K130831].

Methods of the invention may comprise a step consisting of comparing the proteins and fragments concentration in circulating cells with a control value. As used herein, “concentration of protein” refers to an amount or a concentration of a transcription product, for instance the proteins of the invention. Typically, a level of a protein can be expressed as nanograms per microgram of tissue or nanograms per milliliter of a culture medium, for example. Alternatively, relative units can be employed to describe a concentration. In a particular embodiment, “concentration of proteins” may refer to fragments of the proteins of the invention.

As used herein, a “reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the genes expression level (obtained according to the method of the invention) with a defined threshold value. According to the invention, the reference value is the ratio of the expression of the gene after TNF inhibitors treatment versus the expression of non-treated gene. Thus, according to the invention, the reference value is determined by calculating the ratio of the expression of the gene after being stimulated with the TNF inhibitors and the expression of the gene no-stimulated with the TNF inhibitors in patients classified as responders or non-responders.

In some embodiment, the reference value is a value between 0.94 and 1.06.

In another words, in some embodiment, the reference value is 0.94, 0.95, 0.96, 0.97, 0.99, 1.00, 1.01, 1.02, 1.03, 1.04, 1.05 or 1.06.

In some embodiment, the reference value is 0.94 or 1.06

In some embodiments, the method of the invention comprises the use of a classification algorithm typically selected from unsupervised hierarchical clustering, Linear Discriminant Analysis (LDA), Topological Data Analysis (TDA), Neural Networks, Support Vector Machine (SVM) algorithm and Random Forests algorithm (RF). In some embodiments, the method of the invention comprises the step of determining the patient's response to TNF inhibitors using a classification algorithm.

As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

In some embodiments, the method of the present invention comprises a) quantifying the level of plurality of genes listed above in the sample; b) implementing a classification algorithm on data comprising the quantified plurality of genes so as to obtain an algorithm output; c) determining the survival time from the algorithm output of step b).

The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

In some embodiment, the group of genes as disclosed herein is useful for determining responder to anti-TNF antibody therapy.

### Method for Treating Inflammatory Disease

Accordingly, patients identified as anti-TNF antibody responder according to the invention can be administered anti-TNF antibody therapy, for example systematic therapy. Patients identified as anti-TNF antibody non-responder according to the invention can be administered with other classical treatment of inflammatory disease.

Thus, in a second aspect, the invention relates to a method for treating inflammatory disease in a patient in need thereof comprising administering a therapeutically effective amount of anti-TNF antibody when the patient is identified as activators according to the invention.

In some embodiment, a therapeutically effective amount of classical treatment of inflammatory disease is administered when the patient is identified as non-activators according to the invention.

In some embodiment, the TNF inhibitor is a monoclonal antibody.

In some embodiment, the TNF inhibitor is adalimumab, golimumab, infliximad.

In some embodiment, the inflammatory disease is rheumatoid arthritis, crohn's disease, ankylosing spondylitis or psoriatic arthritis.

As used herein, the term “classical treatment of inflammatory disease” has its general meaning in the art and refers to any compound, natural or synthetic, used for the treatment of inflammatory disease.

According to the invention, classical treatment of inflammatory disease is not anti-TNF antibody.

Example of compounds used for the treatment of inflammatory disease include corticosteroids; aminosalicylates such as mesalamine, balsalazide and olsalazine; immunosuppressant drugs such as aziathioprine, mercaptopurine, cyclosporine and methotrexate; aspirin, celecoxib; diclofenac; diflunisal; etodolac; ibuprofen; indomethacin; ketoprofen; Janus kinase (JAK) inhibitor such as ruxolitinin, tofacitinib, oclacitinin, baricitinib, perficitinib, fedranitib, upadacitinib, fligotinin, cerdulatinib, gandotinib, lestaurtinin, momelotinib, pacritinib and abrocitinib; anti-interleukin 6 (anti-IL-6) such as olokizumab, elsilimomab, sirukumab, levilimab, and clazakizumab; agonist of cytotoxic T-lymphocyte-associated protein 4 (CTLA4) such as abatacept and belatacept; ketorolac; nabumetone; naproxen; oxaprozin; piroxicam; salsalate; sulindac; tolmetin; antileukotrienes; antibiotic agents such as penicillin, quinoline, vancomycin, sulfonamides, ampicillin, ciprofloxacin, teicoplanin, telavancin, bleomycin, ramoplanin, decaplanin, chloramphenicol and sulfisoxazole.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “therapeutically effective amount” refers to an amount effective, at dosages and for periods of time necessary, to achieve a desired therapeutic result. A therapeutically effective amount of the immune checkpoint inhibitor of the present invention may vary according to factors such as the disease state, age, sex, and weight of the individual, and the ability of the immune checkpoint inhibitor of the present invention to elicit a desired response in the individual. A therapeutically effective amount is also one in which any toxic or detrimental effects of the antibody or antibody portion are outweighed by the therapeutically beneficial effects. The efficient dosages and dosage regimens for the immune checkpoint inhibitor of the present invention depend on the disease or condition to be treated and may be determined by the persons skilled in the art. A physician having ordinary skill in the art may readily determine and prescribe the effective amount of the pharmaceutical composition required. For example, the physician could start doses of the immune checkpoint inhibitor of the present invention employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of a inhibitor of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a inhibitor of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time.

A third aspect of the invention relates to a therapeutic composition comprising an anti-TNF antibody for use in the treatment of inflammatory disease in a patient identified as activators to said anti-TNF antibody as described above. Thus, the invention relates to a therapeutic composition comprising anti-TNF antibody for use in the treatment of inflammatory disease in a patient identified as responder to said anti-TNF antibody as described above.

The invention also relates to a therapeutic composition comprising a classical treatment of inflammatory disease for use in the treatment of inflammatory disease in a patient identified as non-activator to anti-TNF antibody as described above.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Particularly, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

### Kit for Performing the Method According to the Invention

In a fourth aspect, the invention relates to a kit for performing the methods of the present invention, wherein said kit comprises means for measuring the expression level of CD36, FBX030, GABARA, LBR, MAFG, OSGIN1, HMOX1 and/or CSMD1 in a biological sample.

More particularly, the kit comprising:
(a) at least one reagent for measuring the expression of CD36, FBX030, GABARA, LBR, MAFG, OSGIN1, HMOX1 and/or CSMD1 in a biological sample obtained from a subject, and
(b) instructions for use.

In some embodiment, the kit comprises means for purified monocytes, and in particular CD14+ monocytes.

Thus, the kit comprising:
(a) at least one reagent for measuring the expression of CD36, FBX030, GABARA, LBR, MAFG, OSGIN1, HMOX1 and/or CSMD1 in a biological sample obtained from a subject, and
(b) at least one filter and/or marker method for isolated from blood sample CD14+ monocytes
(c) instructions for use.

The invention will be further illustrated by the following figures and examples. However, these examples and figures should not be interpreted in any way as limiting the scope of the present invention.

### FIGURES

FIG. 1: TNFi are not able to induce a NRF2 translocation in all healthy donors. CD14+ monocytes purified from healthy blood donors were differentiated during 5 days into macrophages in presence of recombinant GM-CSF (20 ng/mL) prior to being stimulated or not (T0) with TNFi (10 μg/mL). After 0.75, 2 or 5 hours of stimulation with certolizumab pegol (Fab) (A) or 2 hours with Fab, TNFi antibody or TNF soluble receptor 2 (B), cells were harvested for cell fractionation. NRF2 and PCNA protein expression in nuclear parts were analyzed by Western-Blot. Blood donors who increases their NRF2 expression level in nucleus were classified NRF2 translocator. (A) NRF2 expression is normalized on PCNA expression. Data represent mean±SEM (n=6 for activators, n=7 for non-activators, ** p<0.01, *** p<0.001, unpaired Student's t test). (B) Western blot data are representative of 5 independent donors.

FIG. 2: identification of target genes indicating a transcriptional activation of tmTNF reverse signaling in healthy donors. CD14+ monocytes purified from healthy blood donors were differentiated during 5 days into macrophages in presence of recombinant GM-CSF (20 ng/mL) prior to being stimulated (T) or not (NT) with TNFi (10 μg/mL) during 16 hours. Cells were harvested and (A) cd36, fbx030, lbr, mafg, (B) gabara osgin1, hmox-1 and csmd1 mRNA expression analyzed by RT-qPCR. Donors were classified as Activators when a gene expression up-regulation was observed in contrary of non-activators. Data are presented as mean±SEM of mRNA fold change vs NT normalized on gapdh. (n=17 for activators and n=13 for non-activators, except for fbx030, n=8 for Activators and n=6 for non-activators and csmd1 n=6, *p<0.05, **p<0.01, paired t-test performed).

FIG. 3: Identification of experimental variation of mRNA measurement and definition of tmTNF reverse signaling activator/non-activator status. (A) Coefficient of variation (CV) of qPCR experiments was measured by repeating the quantification of cd36 of one sample. mRNA expression was analyzed by technical duplicate and repeated 94 times. Coefficient of variation was calculated by dividing the 94 samples mean by their standard deviation. (B) Definition of tm TNF reverse signaling activator status. First of all, CD36 mRNA ratio expression (T/NT) is analyzed and after application of CV, if this ratio is less than 0.94, individual is classified as non-activator. If cd36 ratio is greater than 1.06, an analysis of NRF2 target genes is necessary. If at least 50% of NRF2 target genes ratio are greater than 1.06 then individual is classified as activator. Otherwise, individual is classified as non-activator. (C) Analysis of RT-qPCR replicates variability and its impact on tmTNF reverse signaling status classification. 3 replicates of RT-qPCR were performed with 1 or 2 days of interval for a healthy non activator donor and a healthy activator donor. Application of CVR is represented by a scale cut between 0.94 and 1.06 on y. Data are presented as mean±SEM of mRNA fold change vs NT normalized on gapdh. (n=3).

FIG. 4: Experimental protocol to predict TNFi clinical response depending on tmTNF reverse signaling activator status. RA Patients blood was collected before initiation of TNFi therapy. CD14+ monocytes are purified prior to being stimulated or not with different TNFi (10 μg/mL, soluble receptor, antibody or Fab) during 16 hours. Cells were harvested and cd36, fbx030, gabara, lbr, mafg, osgin1, hmox-1 and csmd1 mRNA expression analyzed by RT-qPCR. Depending on RT-qPCR results, patients are classified as Activators or Non-activators. In parallel, patients are treated with TNFi therapy. After 3 months, clinical response is analyzed by physicians and patients are classified Responder or Non-Responder to TNFi treatment following EULAR criteria (decrease of DAS28 by 1.2 points). Reverse signaling activation status and clinical response are analyzed to verify our prediction accuracy.

FIG. 5: Results of experimental prognostic protocol on 9 RA patients. Results of RT-qPCR analysis of 9 RA patients treated with monoclonal antibody included in our protocol are presented here. Application of CVR is represented by a scale cut between 0.94 and 1.06 on y. Following our prediction protocol, classification and clinical status obtained after 3 months of treatment are indicated below histograms.

### EXAMPLE

Material & Methods

Healthy Donor's Monocytes and Macrophage Differentiation

Healthy donors Monocytes were purified from buffy coat obtained at the EFS (Etablissement Français du Sang, France), layered on Pancoll gradient (PanBiotech, Aidenbach, Germany), and positively isolated by CD14+ magnetic sort following manufacturer's instructions (Life Technologies, CA, USA). Purified monocytes were plated and cultured at a density of 1-2×10⁶ cells/well (12-well plate, Falcon poly-styrene) in Roswell Park Memorial Institute medium (RPMI, Invitrogen, CA, USA) supplemented with 10% heat-inactivated fetal bovine serum (FBS) (Life Technologies), 1% penicillin/streptomycin (Invitrogen) and recombinant GM-CSF (20 ng/mL, peprotech) during 5 days of culture in presence of recombinant human GM-CSF (Peprotech, NJ, USA) to induce their differentiation into macrophages prior to being stimulated 16 hours with 10 μg/mL of TNFi (Adalimumab, Humira Abbvie; Certolizumab Pegol, Cimzia, UCB; Etanercept, Enbrel, Pfizer; Golimumab, Simponi, Janssen; Infliximab, Remicade, Janssen).

Quantitative Real-Time PCR

Macrophages were harvested after 5 days of differentiation and 16 hours of culture in presence of TNFi. RNA was extracted by using High Pure RNA Isolation Kit (Roche, Switzerland) and reverse transcribed with iscript cDNA synthesis kit (Bio-Rad, CA, USA) according to the manufacturer's instructions. All qPCRs were performed with SYBR green mastermix (Bio-Rad).

Cell Fractionation and Western Blot Analysis

Macrophages were harvested after 5 days of differentiation and 2 hours of culture in presence of TNFi. Cells were washed two times with cold PBS and centrifuged. Pellets were resuspended in 500 IA of cytop Buffer (Triton 0.25%, Tris HCl 10 mM, EDTA 5 mM, EGTA 0.5 mM and proteases inhibitor cocktail) and incubated 3-5 min on ice. After centrifugation, supernatant was kept as soluble part (cytoplasm) and pellet were lysed with TNEN 250:0.1 buffer (NaCl 250 mM, TrisHCl 50 mM, EDTA 5 mM, NP40 0.1% and proteases inhibitor cocktail) 20-30 min on ice. After centrifugation, supernatants were kept as insoluble part (nucleus) and pellets were lysed in Urea 8 M, 10 min at 95° C. to recover chromatin part. Total amount of protein was quantified by BCA assay.

Nuclear lysates were then subjected to SDS/PAGE on 4-12% polyacrylamide gels (ThermoFisher scientific, Walham, MA, USA). After transferring on 0.22 μm nitrocellulose membrane, proteins were revealed using NRF-2 (D1Z9C, 04/2018, Cell Signaling Technology) and PCNA (PC10, sc-56, Santa Cruz Biotechnologies, CA, USA) specific antibodies and anti-rabbit or anti-mouse HRP-linked polyclonal antibodies (Cell Signaling, MA, USA).

Flow Cytometry Analysis

Macrophages used for cell surface staining were washed with PBS-EDTA 5 mM, PBS-SVF 5% and stained with monoclonal Mouse IgG1 anti-tmTNF PE-conjugated antibodies (FAB210P, R&D systems, Minneapolis, MN, USA) or anti-CD36 PE-conjugated (555455, Becton Dickinson, NJ, USA) in presence of FcR-blocking reagent (Miltenyi biotech, Germany). Flow cytometry analyses were performed with a MACSQuant analyzer 10 flow cytometer (Miltenyi biotech). Data were analyzed with FlowJo software.

Patients

RA patients starting an TNFi therapy as a first lign were selected (Consent, BIOTOUL). All patients received Methotrexate as a background treatment and less than 10 mg of corticoids.

Predicting Protocol of Clinical Response to TNFi

RA patients blood were collected prior their TNFi primo-injection. Monocytes were positively isolated by CD14+ magnetic sort following manufacturer's instructions (Life Technologies, CA, USA) and plated at a density of 1×10⁶ cells/well (12-well plate) in RPMI (Invitrogen) supplemented with 10% heat-inactivated FBS (Life Technologies) and 1% penicillin/streptomycin (Invitrogen) prior to being stimulated 16 hours or not with TNFi (10 μg/mL). All kind of TNFi (Antibody, Fab and soluble receptor) were used in different wells to study their own efficiency to activate tmTNF reverse signaling in patient's monocytes. mRNA levels were analyzed by RT-qPCR. CD36 and tmTNF expression was assessed by flow cytometry before and after in vitro TNFi treatment. After in vitro results analysis, patients are classified as tmTNF reverse signaling activators or non-activators. After 3 months of treatment, physicians analyze patient's clinical response and classified them as responder or non-responder to TNFi therapy, following EULAR criteria (a DAS28 down-regulation superior of 1.2 points). Finally, tmTNF reverse signaling activation status and clinical response are compared to verify our prediction test accuracy.

Statistical Analysis

All data were analyzed with GraphPad Prism5. Normality was tested by Agostino and Pearson test. In vitro data were analyzed with Student's T-test or Mann-Whitney U-test. Data are represented as mean±SEM, and p<0.05 (two-tailed) was considered to be statistically significant.

Results

TNFi are not Able to Induce a NRF2 Translocation in all Donors.

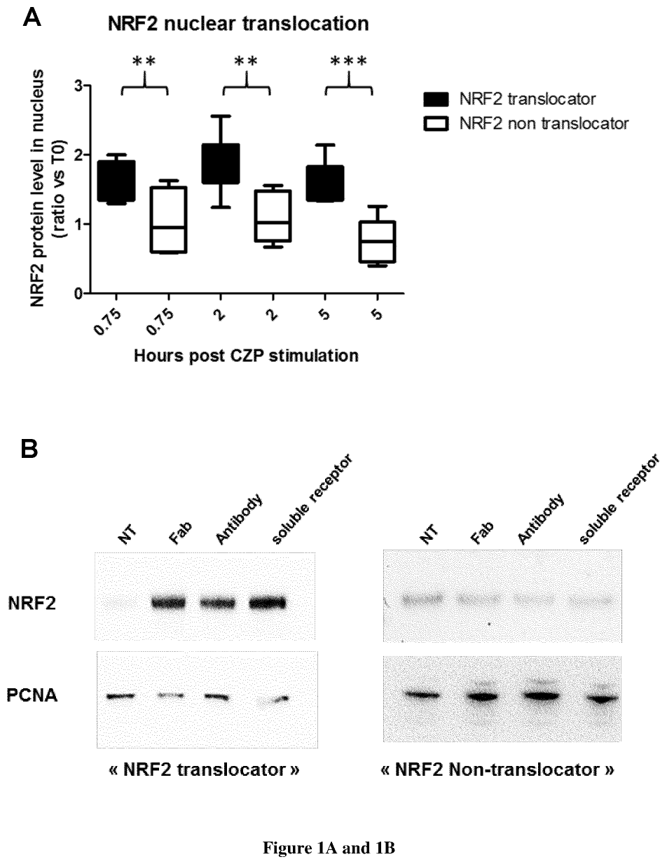

First, we assessed whether the nuclear translocation of NRF2 after stimulation with certolizumab pegol (CZP, an TNFi) observed in monocytes could also be observed in macrophages. For this, purified CD14+ monocytes from healthy donors (EFS) were differentiated 5 days into macrophages in presence of GM-CSF prior stimulation by CZP in vitro. We were able to confirm that stimulation of macrophages by CZP induces a nuclear translocation of NRF2 after 0.75 h and maintained even after 5 hours of stimulation (FIG. 1A grey histogram, NRF2 translocator). Interestingly, we could observe that some donors did not display this NRF2 nuclear translocation after CZP stimulation (FIG. 1A, white histograms, NRF2 non translocator). We further analyzed this NRF2 nuclear translocation after stimulation with the 3 different types of TNFi: an antibody or a soluble receptor. We also distinguished two profiles with NRF2 translocators and NRF2 non-translocators with all types of TNFi molecules (FIG. 1B).

Identification of Target Genes Indicating a Transcriptional Activation of tmTNF Reverse Signaling in Healthy Donors.

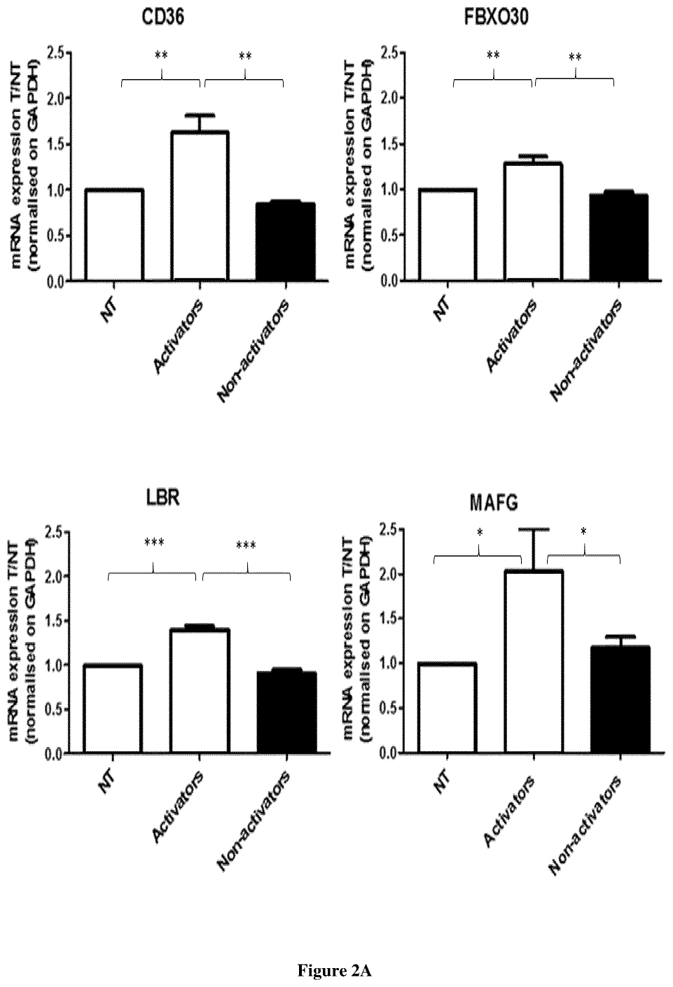

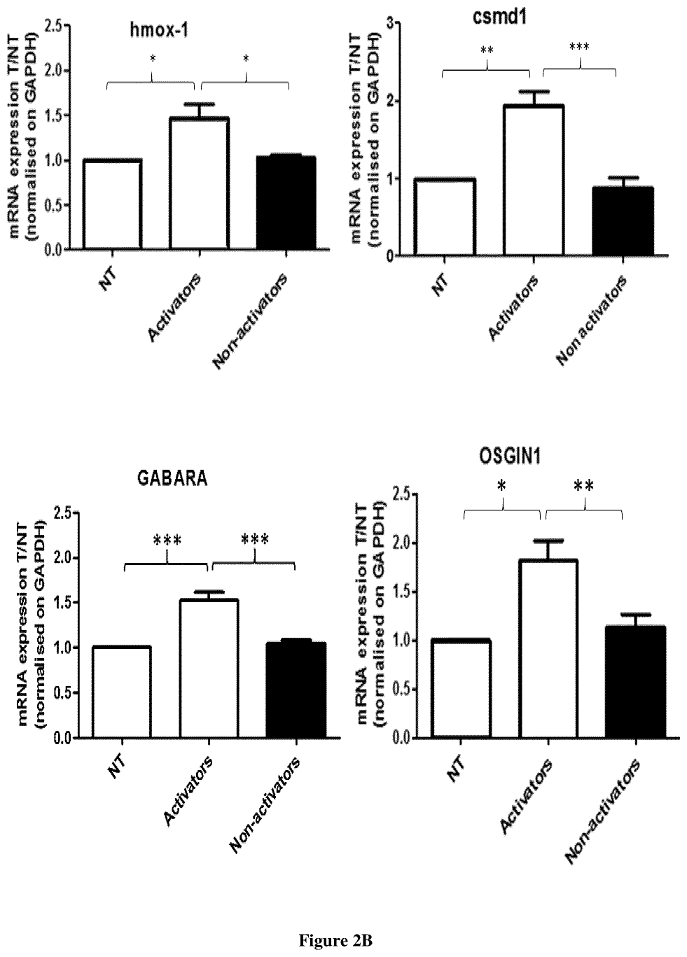

To go further in our observations, we analyzed the transcriptional activity of NRF2 in macrophages of healthy donors after TNFi stimulation. To this end, we evaluated by RT-qPCR the mRNA expression of CD36, which was already described to be regulated by TNFi 5, some specific NRF2 target genes (FBX030, GABARA, LBR, MAFG, OSGIN1, HMOX1), which play a role in the anti-oxidative stress response or anti-inflammatory pathway, and the expression of CSMD1, an anti-inflammatory gene that we observed as up-regulated by all TNFi in our laboratory (data not published). Interestingly, we observed 2 different subsets of healthy donors: (i) donors in which TNFi stimulation increased mRNA of these target genes in macrophages (FIG. 2, white bars), ‘(ii) and conversely donors with no significant upregulation in transcription of these target genes (FIG. 2, black bars). Based on these observations, we classified donors into two different status, “activators” or “non-activators” of tmTNF reverse signaling after TNFi stimulation.

As tmTNF reverse signaling may play an important role in the therapeutic response to TNFi during arthritis, we hypothesize that “non-activators” of tmTNF reverse signaling correspond to the clinically non-responders to TNFi.

Identification of Experimental Variation of mRNA Measurement and Definition of tmTNF Reverse Signaling Activator/Non-Activator Status.

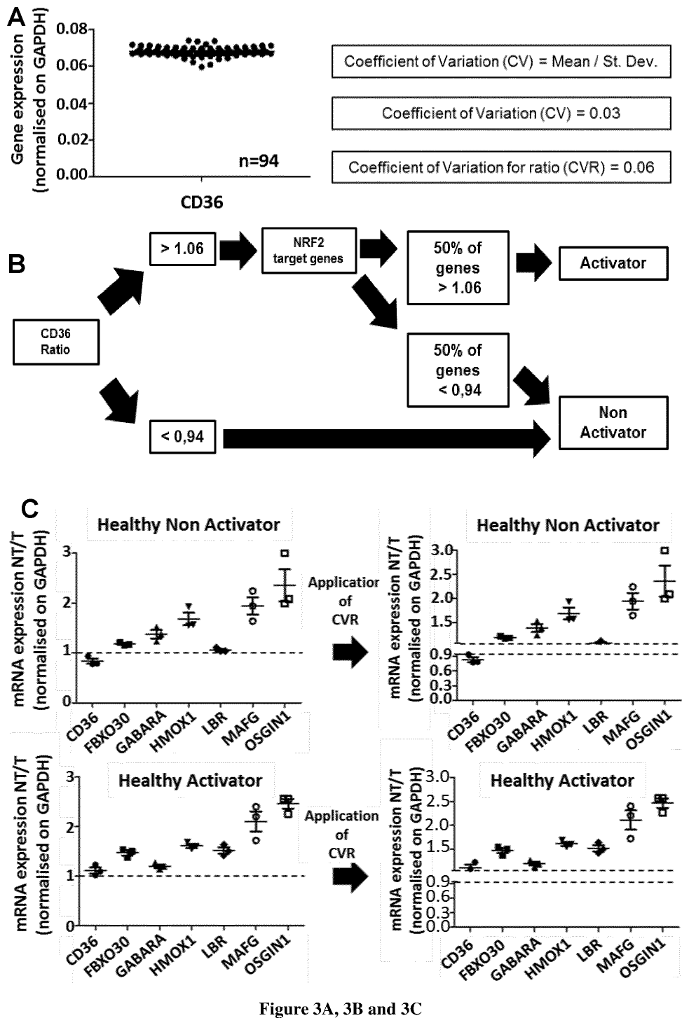

To optimize evaluation of the prognostic value of the RT-qPCR analysis of tmTNF reverse signaling activation and our classification into activators or non-activators, we calculated the coefficient of variation (CV) of our RT-qPCR measurements. To this end, we repeated 94-fold the analysis of mRNA expression of different genes (CD36, HMOX1). Coefficient of variation was then calculated by dividing the mean by the standard deviation of these 94 measurements and we obtained a CV of 0.03 (FIG. 3A). To determine the variation of a target gene, we calculate the ratio of its expression after TNFi treatment versus the non-treated one. As we use a ratio calculation, our CV was then 0.06 for these results.

Based on all these observations, we developed a protocol to classify donors or patients into activators or non-activators of tmTNF reverse signaling after TNFi stimulation. After 16 hours of TNFi treatment, we first analyzed the CD36 mRNA ratio (Treated vs NT). If this ratio is lower than 0.94 (lower than 1 after application of our CV), then the donor/patient is classified as non-activator. If this ratio is upper than 1.06 (upper than 1 after application of our CV), then we analyzed the modulation of 6 NRF2 target genes (GABARA, FBX030, HMOX1, MAFG, LBR, OSGIN1). If at least 50% of these NRF2 target genes are up-regulated more than 1.06, then the donor/patient is classified as tmTNF reverse signaling activator. On the contrary the donor/patient is classified as non-activator (FIG. 3B). Finally, following this protocol, we analyzed the effect of our RT-qPCR machine variation on the classification. To this end, we repeated the RT-qPCR experiments 3 times, on different days and after frost and defrost cycles. In FIG. 3C, we observed that technical variation was not biologically significant and did not impact patient/donor classification.

Experimental Protocol to Predict TNFi Clinical Response Depending on tmTNF Reverse Signaling Activator Status.

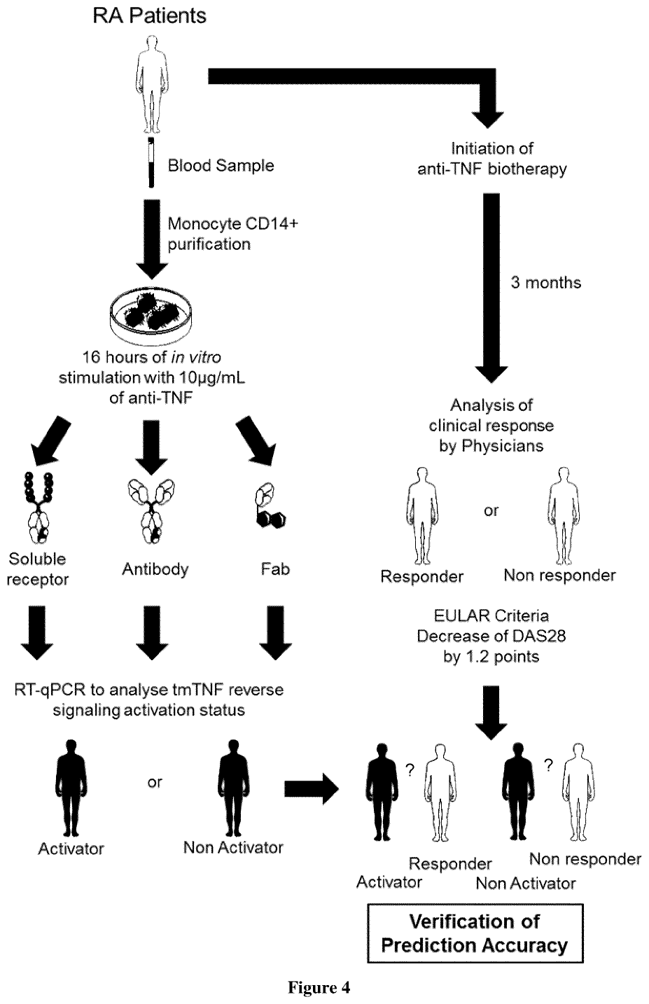

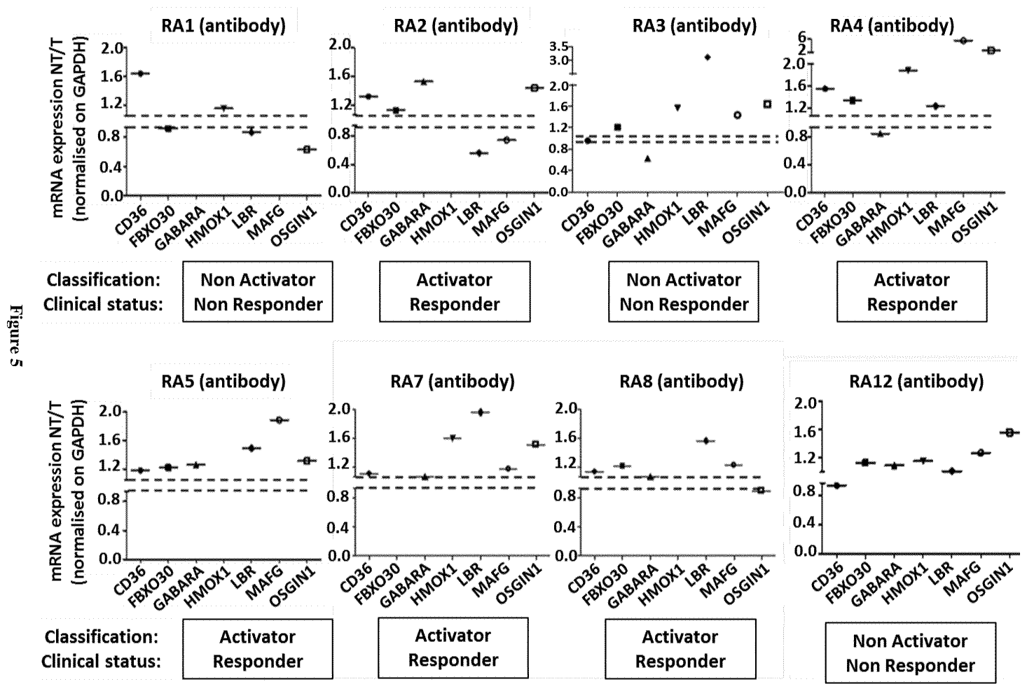

Then, we developed an experimental protocol to predict TNFi clinical response through the determination of tmTNF reverse signaling activator/non-activator status. Blood samples from RA patients were collected before initiation of TNFi therapy. CD14+ monocytes were purified prior being stimulated or not with the TNFi initiated in real-life (10 μg/mL, soluble receptor, antibody or Fab) during 16 hours. Cells were harvested and cd36, fbx030, gabara, lbr, mafg, osgin1, hmox-1 and csmd1 mRNA expression analyzed by RT-qPCR. Depending of RT-qPCR results, patients were classified as Activators or Non-activators. After 3 months, clinical response was evaluated by physicians and patients were classified Responder or non-responder to TNFi treatment following EULAR criteria. Baseline reverse signaling activation status was compared to the real-life month-3 clinical response to assess prediction accuracy (FIG. 4).

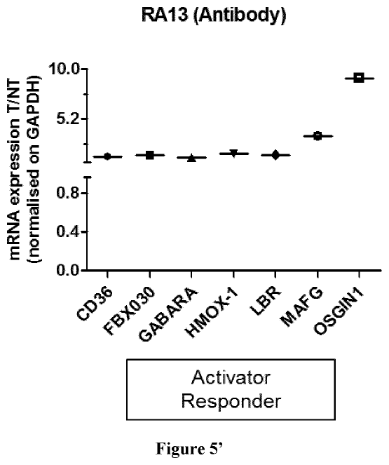

Until now, we were able to include 9 RA patients in our study treated with antibodies. All patients received Methotrexate as a background treatment and less than 10 mg of corticoids (Prednisone). FIG. 5A shows the results of RT-qPCR analysis of target genes transcriptional variation after in vitro stimulation of patients purified monocytes. Based on our activator classification previously described and after the analysis of patient's clinical response we were able to predict the clinical response to TNFi monoclonal antibody with a 100% accuracy. Moreover, we need to integrate more patients treated with Fab or soluble receptor and also other target genes which will be more specific to Fab and soluble receptor mediated transcriptional modulation.

### REFERENCES

Throughout this application, various references describe the state of the art to which this invention pertains. The disclosures of these references are hereby incorporated by reference into the present disclosure.
1. Daien, C. et al. Update of French society for rheumatology recommendations for managing rheumatoid arthritis. Joint Bone Spine 86, 135-150 (2019).
2. Smolen, J. S. et al. EULAR recommendations for the management of rheumatoid arthritis with synthetic and biological disease-modifying antirheumatic drugs: 2019 update. Ann Rheum Dis annrheumdis-2019-216655 (2020) doi:10.1136/annrheumdis-2019-216655.
3. Eissner, G. et al. Reverse signaling through transmembrane TNF confers resistance to lipopolysaccharide in human monocytes and macrophages. J Immunol 164, 6193-8 (2000).
4. Zhang, Z. et al. Transmembrane TNF-alpha promotes chemoresistance in breast cancer cells. Oncogene 37, 3456-3470 (2018).

Boyer, J. F. et al. Anti-TNF certolizumab pegol induces antioxidant response in human monocytes via reverse signaling. Arthritis Res Ther 18, 56 (2016).
6. Ahmed, S. M., Luo, L., Namani, A., Wang, X. J. & Tang, X. Nrf2 signaling pathway: Pivotal roles in inflammation. Biochim Biophys Acta 1863(2), 585-597 (2016).
7. Diallo, K. Demonstration in vivo de la signalisation inverse du TNF transmembranaire dans la réponse thérapeutique aux molécules anti-TNF dans un modèle d'arthrite murine. (2019).
8. Ebrahimi, A. A. et al. Serum levels of TNF-alpha, TNF-alphaRI, TNF-alphaRII and IL-12 in treated rheumatoid arthritis patients. Iran J Immunol 6, 147-153 (2009).
9. Taylor, P. C. Pharmacology of TNF blockade in rheumatoid arthritis and other chronic inflammatory diseases. Current Opinion in Pharmacology 10, 308-315 (2010).

1. An in vitro method for classifying a patient suffering from an inflammatory disease as an anti-TNF antibody activator and treating the patient, comprising the steps of:
i) determining, in a sample obtained from the patient, the expressions levels of a CD36 gene with and without stimulation with anti-TNF antibody, and
ii) determining in the sample obtained from the patient, the expressions levels of at least two genes selected from the group consisting of: FBX030, GABARA, LBR, MAFG, OSGIN1, HMOX1 and CSMD1, wherein the expression levels of each of the at least two genes are determined with and without stimulation with the anti-TNF antibody,
iii) treating, with the anti-TNF antibody, the patient identified as having a between the expression levels of CD36 with and without stimulation that is higher than a corresponding reference value and as having at least one ratio of the expression levels, with and without stimulation with the anti-TNF antibody, of at least one of the at least two genes determined at step ii) that is higher than a corresponding reference value.

2. (canceled)

3. The in vitro method according to claim 2, wherein the expression level of 2, 3, 4, 5, 6 or 7 genes selected from the group consisting of: FBX030, GABARA, LBR, MAFG, OSGIN1, HMOX1 and CSMD1 are determined with and without stimulation with the anti-TNF antibody.

4. The in vitro method according to claim 2, wherein the expression level of FBX030, GABARA, LBR, MAFG, OSGIN1 and HMOX1 are determined with and without stimulation with the anti-TNF antibody.

5. The in vitro method according to claim 2, wherein, for the patient that is treated, at least 50% of the ratios are higher than the corresponding reference values.

6. (canceled)

7. The in vitro method according to claim 1, wherein the inflammatory disease is rheumatoid arthritis, Chron's disease, ankylosing spondylitis or psoriatic arthritis.

8. The in vitro method according to claim 1, wherein the patient has been previously treated with methotrexate.

9. The in vitro method according to claim 1, wherein the sample is a blood sample.

10. The in vitro method according to claim 1, wherein the corresponding reference value is between 0.94 and 1.06.

11. (canceled)

12. (canceled)

13. (canceled)

14. A therapeutic composition comprising an anti-TNF antibody.

15. A kit for performing the methods of the present invention, wherein said kit comprising:
(a) at least one reagent for measuring the expression of CD36, FBX030, GABARA, LBR, MAFG, OSGIN1, HMOX1 and/or CSMD1 in a biological sample obtained from a subject, and
(b) instructions for use.

16. The in vitro method according to claim 9, wherein the blood sample is a peripheral blood mononuclear cell (PBMC) sample.
